(19) United States
(12) Patent Application Publication
(10) Pub. No.: US 2026/0165841 A1
(43) Pub. Date: Jun. 18, 2026

(54) COMPOSITE SANDWICH STRUCTURE OF ORTHOPEDIC IMPLANT

(71) Applicant: Industrial Technology Research Institute, Hsinchu (TW)

(72) Inventors: Chih-Chieh HUANG, Zhunan Township (TW); Shih-Ping LIN, Hengshan Township (TW); Pei-I TSAI, Hsinchu City (TW); Yuchi WANG, New Taipei City (TW); Kuo-Kuei HUANG, Zhubei City (TW)

(73) Assignee: Industrial Technology Research Institute, Hsinchu (TW)

(21) Appl. No.: 18/999,884

(22) Filed: Dec. 23, 2024

(30) Foreign Application Priority Data
Dec. 18, 2024 (TW) 113149375

(51) Int. Cl. A61F 2/28

(52) U.S. Cl. CPC A61F 2/28; A61F 2002/2835; A61F 2250/0063; A61F 2310/00155; A61F 2310/00179; A61F 2310/00329; A61F 2310/00982

(57) ABSTRACT

A composite sandwich structure of orthopedic implant is provided, which includes: a core, a reinforcement layer covering the outer surface of the core, and an affinity layer further covering the outer surface of the reinforcement layer. The material of the core includes polyaryletherketone (PAEK), the reinforcement layer includes at least one braided layer composed of carbon fiber bundles, and the affinity layer includes PAEK and/or compounded further with bio-ceramics.

### CROSS REFERENCE TO RELATED APPLICATIONS

This Application claims priority of Taiwan Patent Application No. 113149375, filed on Dec. 18, 2024, the entirety of which is incorporated by reference herein.

### TECHNICAL FIELD

The present invention relates to composite sandwich structure of orthopedic implant, and in particular, it relates to the composite sandwich structure of orthopedic implant of polyaryletherketone material covered with carbon fiber bundles.

### BACKGROUND

An ideal material for bone grafts should have mechanical properties that are similar to those of hard bone, such as an elastic coefficient of about 14.1-27.6 GPa, and a tensile strength of about 150 MPa. Commonly used implants today can be roughly divided into two categories: metal and non-metal. However, the most common metal bone materials used these days (such as Ti-6A1-4V, with an elastic modulus of about 95-117.4 GPa and a tensile strength of about 900 MPa) have high mechanical strength, but their stiffness is too high to match with the bone mechanics of human body. As a result, the human bone tissue is unable to withstand sufficient force over a long period of time, resulting in stress shielding effect that leads to bone atrophy and resorption, and collapse of the bone tissue structure. On the other hand, polymers as non-metallic materials (such as PEEK, with an elastic modulus of about 3-4 GPa and a tensile strength of about 50 MPa) have a moderate mechanical strength compared to metal bone materials, and they are more similar to the mechanical properties of human bones. Still, they are insufficient to bear high-loading.

In addition, patients with spinal tumors need to receive radiation therapy after tumor resection surgery today. During tumor resection surgery, after the vertebrae eroded by cancer cells are removed, the upper and lower vertebrae need to be fixed into place with pedicle screws to maintain the stability of the spine. However, when acquiring radiological medical images, metal implants may cause image scattering, and can cause damage to normal tissues during radiotherapy due to an inability to locate the tumor precisely. Furthermore, metal shielding may attenuate the radiation energy and prevent the radiation therapy from achieving the desired therapeutic effect. Although non-metallic implants do not have the problems of image scattering and energy attenuation, most non-metallic spinal implants currently commercially available are made of polyetheretherketone (PEEK) materials, and spinal fixation requires higher mechanical strength. Therefore, this type of implant used in spinal fixation is often at risk of fracture and damage, causing patients to undergo reoperation or other complications.

Although overseas manufacturers have, in recent years, developed orthopedic materials using micron-grade carbon fibers mixed with PEEK to enhance the mechanical strength of PEEK, the carbon fibers have changed the material properties of the PEEK polymers so that the ductility of this kind of material has decreased from about 15% to less than 1% of that of PEEK. Therefore, this material is hard and brittle, and difficult to use. In addition, there is a risk of particles releasing from the carbon fiber causing an inflammatory reaction.

Therefore, there is currently still a lack of non-metallic biomedical materials with both high mechanical strength and ductility in clinical practice.

### SUMMARY

In view of the above problems, the present disclosure provides a composite sandwich structure of orthopedic implant, using a reinforcing layer that includes a carbon fiber bundle braided layer to reinforce a non-metallic material. In addition, through a variable braided structure, the present disclosure is able to adjust the mechanical properties of the implant by using different braided structures at different portions of a single implant to match the mechanical requirements.

An embodiment of the present disclosure provides a composite sandwich structure of orthopedic implant, which includes: a core with material being polyaryletherketone (PAEK), a reinforcement layer covering the surface of the core, and an affinity layer covering the surface of the reinforcement layer. The reinforcement layer includes at least one braided layer composed of carbon fiber bundles, and the affinity layer includes PAEK and/or composite material of bio-ceramics.

### BRIEF DESCRIPTION OF THE DRAWINGS

The following embodiments of the present disclosure are described in detail with the accompanying figures. It is emphasized that, in accordance with the standard practice in the industry, various features are not drawn to scale. In fact, the dimensions of the various features may be arbitrarily increased or reduced for clarity of discussion.

FIGS. 1A to 1D are schematic cross-sectional views of composite sandwich structures of orthopedic implants, according to some embodiments of the present disclosure.

FIGS. 2A to 2D are schematic cross-sectional views of composite sandwich structures of orthopedic implants, according to some other embodiments of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number.

The present disclosure uses a reinforcement layer, having a carbon fiber bundle braided layer, to reinforce the high-performance polymer as the core of the orthopedic implant, and covers an affinity layer, comprising a polyaryletherketone (PAEK) material, as the outermost layer of the orthopedic implant. In addition to preventing inflammatory reaction caused by the exposure of the carbon fiber, and improving biocompatibility, the original mechanical properties of the PAEK material can be retained to prevent the excessively reduction of ductility that would make the material become hard and brittle.

In addition, literature shows that different portions of the human body have different biomechanical requirements for implants. For example, the proximal end of the femoral bone plate is subjected to much larger force than the distal end. Therefore, the present disclosure develops a variable braided structure, which enables the using of different braided structures at different portions of the same implant to adjust the mechanical properties, so that the implant has the desired mechanical properties.

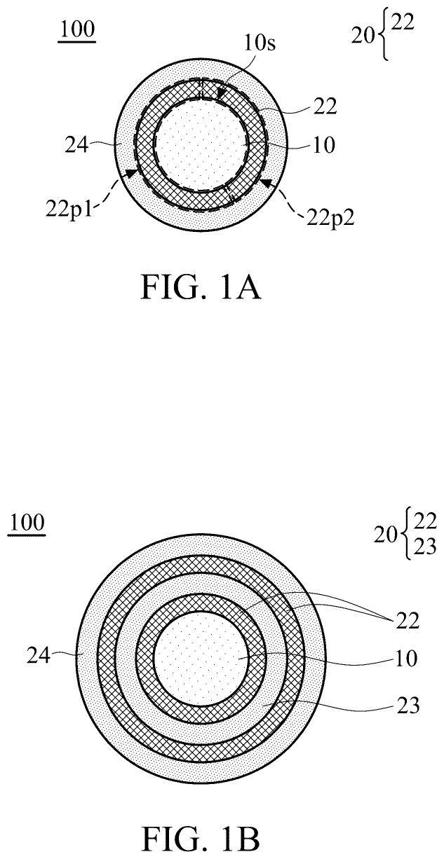

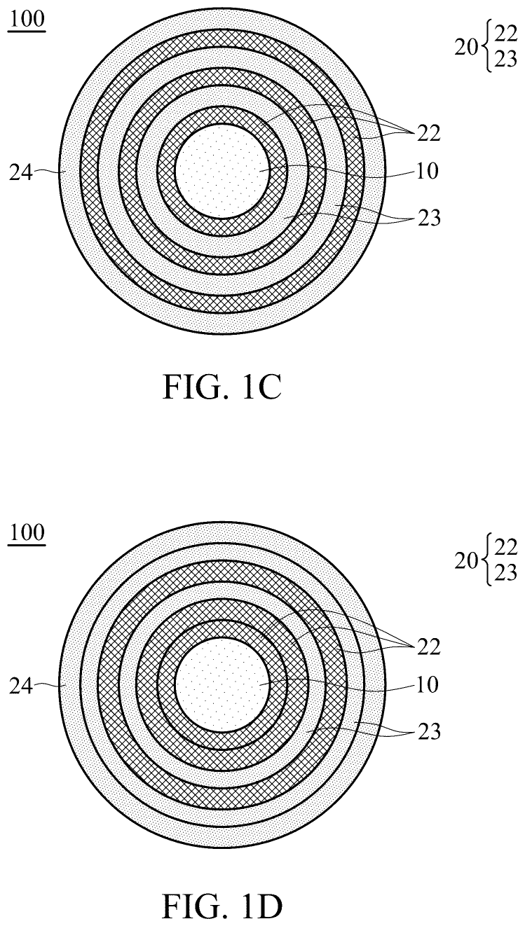

Referring to FIG. 1A, a cross-sectional view of a composite sandwich structure of orthopedic implant 100 is shown, according to some embodiments of the present disclosure. The composite sandwich structure of orthopedic implant 100 includes: a core 10, a reinforcement layer 20 covering the surface of the core 10, and an affinity layer 24 covering the reinforcement layer 20 or the outermost braided layer 22, wherein FIG. 1A only shows that the reinforcement layer 20 only includes a single braided layer 22.

In the present disclosure, polyaryletherketone (PAEK) series materials are used as the core 10. PAEK is a high-performance thermoplastic engineering plastic family with excellent mechanical properties, thermal stability, chemical resistance and biocompatibility. Examples of PAEK that may be used as the core 10 of the present disclosure include polyetherketone (PEK), polyetheretherketone (PEEK), polyetherketoneketone (PEKK), polyetheretherketoneketone (PEEKK), polyetherketoneetherketoneketone (PEKEKK), etc. The size and shape of the core 10 may be adjusted to meet implant requirements.

In some embodiments, the material of core 10 may further mixed with short fibers or staple of carbon fibers. The core 10 may include about 0%-35% by weight of carbon staple fiber, such as about 3%-33%, about 5%-28%, about 10%-25%, about 13%-20%, or about 15%-18% by weight, etc. By containing carbon staple fiber within the core 10 in the above range, the core tensile strength, bending strength, etc. of the composite sandwich structure of orthopedic implant 100 can be improved. In some embodiments, the core 10 may be manufactured by injection molding or CNC processing.

Next, referring to FIG. 1A, a reinforcement layer 20 is formed to cover the surface of the core 10. The reinforcement layer 20 includes at least one braided layer 22. The braided layer 22 in the present disclosure is formed by carbon fiber bundles, and the outermost braided layer 22 is covered with an affinity layer 24 of PAEK material to prevent the exposure of carbon fibers. The number and order of the layers constituting the reinforcement layer 20 are not limited particularly. In some embodiments, the braided layer 22 is disposed at the innermost layer on the core 10. The affinity layer 24 constitutes the outermost layer of the composite sandwich structure of orthopedic implant 100. In some embodiments, the innermost braided layer 22 directly contacts the core 10.

The number of fiber bundles of the carbon fiber bundles used in the braided layer 22 is not limited particularly, and each carbon fiber bundles may have 1,000 to 12,000 carbon fibers, that is, the K number of the carbon fiber bundle may be 1-12, for example, 2-11, 3-10, 4-9, 5-8, 6-7, etc. By having the number of fibers of the carbon fiber bundle in the above range, the mechanical strength of the core 10 may be enhanced. The average fiber diameter of the carbon fibers included in the carbon fiber bundle is not limited particularly, and may be, for example, 1-10 μm, 2-9 μm, 3-8 μm, 4-7 μm, 5-6 μm, etc. By having the average fiber diameter of the carbon fibers of the carbon fiber bundle in the above range, the mechanical strength of the core 10 may be enhanced. In some embodiments, the thickness of each braided layer 22 formed may also enhance the mechanical strength of the core 10, and none of which is limited herein.

The braided layer 22 may be formed on the core 10 using a radial composite braiding device. The radial composite braiding equipment is a circular braiding technology and machine that can well braid the braided material on a 3D curved surface. Therefore, the core 10 of the present disclosure can have different three-dimensional shapes, such as cube, cone, pyramid, cylinder, sphere, ellipsoid, hemisphere, tetrahedron, triangular prism, quadrangular prism, hexagonal prism, irregular shape, etc., and even if the core 10 has an irregular shape, the braided layer 22 can still be formed conformably on the core 10. In some embodiments, the feed rate during braiding may be about 1-50 mm/s, for example, about 5 -45 mm/s, about 10-40 mm/s, about 12-40 mm/s, about 15-35 mm/s, about 18-30 mm/s, about 20-25 mm/s, etc. The rotation speed may be about 10 rpm-100 rpm, for example, about 15-95 rpm, about 20-90 rpm, about 25-85 rpm, about 30-80 rpm, about 35-75 rpm, about 40-70 rpm, about 45-65 rpm, about 50-60 rpm, about 52-55 rpm, etc. The braiding angle may be about 20 degrees-80 degrees, for example, about 20 degrees- 75 degrees, about 25 degrees-70 degrees, about 30 degrees-65 degrees, about 35 degrees-60 degrees, about 40 degrees-55 degrees, about 45 degrees-50 degrees, etc. The braiding parameters during the formation of each braided layer 22 may be the same or different.

About 0-99% of the upper surface of the structure underlying the braided layer 22 may be covered by the braided layer 22. For example, in the embodiment shown in FIG. 1A, about 0-99% of the surface 10s of the core 10 may be covered by the braided layer 22. In other words, the braiding coverage of the braided layer 22 may be about 0-99%, for example, about 5-98%, about 10-95%, about 15-93%, about 20-91%, about 25-90%, about 30-85%, about 35-80%, about 40-75%, about 45-70%, about 50-65%, about 55-60%, etc. The braiding coverage of each braided layer 22 to the underlying structure may be the same or different.

The braided layer 22 is a variable braided structure, that is, when braiding each layer of the braided layer 22, the braiding parameters such as the braiding angle may not be maintained consistently, and different portions of each braided layer 22 may also have different braiding coverage. In other words, each braided layer 22 may independently have one or more braiding angle and one or more braiding coverage. For example, referring to FIG. 1A, in some embodiments, the braiding angle of the first portion 22p1 of the braided layer 22 may be the same as or different from the second portion 22p2 of the braided layer 22, and the braiding coverage of the first portion 22p1 of the braided layer 22 may be the same as or different from the second portion 22p2 of the braided layer 22. In the present disclosure, the braiding parameters and braiding coverage of different portions of each braided layer 22 may be adjusted to meet usage requirements, so that the composite sandwich structure of orthopedic implant 100 is locally strengthened, thereby expanding the application range of the composite sandwich structure of orthopedic implant 100.

After the braided layer 22 is formed, the material of the core 10 may be partially melted and infiltrated into the seams of the braided layer 22 by hot pressing, so that the braided layer 22 is better fixed on the core 10. The temperature of hot pressing may be about 350° C.-500° C., for example, about 360° C.-490° C., about 370° C.-480° C., about 380° C.-470° C., about 390° C.-460° C., about 400° C.-450° C., about 410° C.-440° C., about 420° C.-430° C., etc. The duration of hot pressing may be about 1-10 minutes, such as about 2-9 minutes, about 3-8 minutes, about 4-7 minutes, about 5-6 minutes, etc. The cylinder pressure may be about 50-100 psi, such as about 55-95 psi, about 60-95 psi, about 65-90 psi, about 70-88 psi, about 73-85 psi, about 75-82 psi, about 78-80 psi, etc. After hot pressing, the temperature may be lowered to about 200° C.-300° C. and the pressure may be maintained for, for example, about 1-10 minutes to avoid warping. For example, the temperature may be lowered to about 210° C.-290° C, about 220-280° C., about 230-270° C., about 240-260° C., about 250-255° C., etc., and the pressure may be maintained for, for example, about 2-9 minutes, about 3-8 minutes, about 4-7 minutes, about 5-6 minutes, etc. Next, after the pressure and temperature are slowly lowered to normal, the sample may be removed from the hot presser, and subsequent trimming processing may be conducted according to requirements. By controlling the parameters of hot pressing appropriately, the braided layer 22 and the underlying material, such as the material of the core 10, may be fully intermixed with each other, and material overflow and deformation may be avoided.

Next, the affinity layer 24 is formed on the braided layer 22. The material of the affinity layer 24 may include PAEK alone, and may use PAEK series materials such as PEK, PEEK, PEKK, PEEKK, PEKEKK, etc., or a composite material mixed with bioceramic materials such as bioglass, calcium phosphate, calcium sulfate, etc. In some embodiments, the affinity layer 24 may be coated on the braided layer 22 by injection molding, e.g., by micro injection molding of biomedical polymers, made of highly biocompatible materials, and coated by a material, such as collagen, to enhance the bioaffinity after implantation. The composite sandwich structure of orthopedic implant 100 may have one or more affinity layer 24, and the thickness of each affinity layer 24 may be adjusted to meet requirements.

In the present disclosure, according to the usage requirements, the above-mentioned braiding, hot pressing, and injection molding steps may be repeated arbitrarily to form a reinforcement layer 20 having a plurality of braided layers 22 and a plurality of structural layers 23, as shown in FIG. 1B to FIG. 1D. The material of the structural layer 23 is similar to that of the affinity layer 24 in that both include polyaryletherketone (PAEK), but structural layer 23 does not include bioceramics or collagen, etc. The number and order of layers constituting the reinforcement layer 20 are not limited particularly. For example, the braided layer 22, the structural layer 23 and the braided layer 22 may be formed alternately, an affinity layer 24 may be formed on the outermost layer after forming a single layer or continuous alternating layers of multiple braided layers 22 and structural layers 23, and a braided layer 22 may be formed inserting between each layer of the multiple structural layer 23 to facilitate the melting of hot pressing. The number of layers of the braided layer 22 and the structural layer 23 may be the same or different. When a plurality of braided layers 22 are formed continuously, the hot pressing may be performed before or after the final coating with the affinity layer 24, or the hot pressing may be performed once after each braided layer 22 is formed.

In some embodiments, the reinforcement layer 20 may include alternately formed braided layer 22 and structural layers 23. For example, as shown in FIG. 1B, the reinforcement layer 20 may have two braided layers 22 and one structural layer 23, and the core 10 is sequentially coated with the first braided layer 22 as the innermost layer of the reinforcement layer 20, the first structural layer 23, the second braided layer 22 as the outermost layer of the reinforcement layer 20, and the affinity layer 24 covering the outermost surface of the reinforcement layer 20, thereby forming a five-layer structure of the composite sandwich structure of orthopedic implant 100. The reinforcement layer 20 may also have three braided layers 22 and two structural layers 23. For example, as shown in FIG. 1C, the core 10 is sequentially coated with a first braided layer 22 as the innermost layer of the reinforcement layer 20, a first structural layer 23, a second braided layer 22, a second structural layer 23, a third braided layer 22 as the outermost layer of the reinforcement layer 20, and an affinity layer 24 covering the outermost surface of the reinforcement layer 20, thereby forming a seven-layer structure of a composite sandwich structure of orthopedic implant 100. When the braided layer 22 and the structural layer 23 are alternately formed, the hot pressing may be conducted once after each braided layer 22 is formed.

In some embodiments, the braided layer 22 in the reinforcement layer 20 may be braided repeatedly for multiple layers and then covered with a single-layer or multi-layer structural layer 23. For example, as shown in FIG. 1D, the core 10 is sequentially coated with a first braided layer 22 as the innermost layer of the reinforcement layer 20, a second braided layer 22, a first structural layer 23, a third braided layer 22, a second structural layer 23 as the outermost layer of the reinforcement layer 20, and an affinity layer 24 covering the outermost surface of the reinforcement layer 20, thereby forming a seven-layer structure of an composite sandwich structure of orthopedic implant 100. In some embodiments, no hot pressing is performed after forming the first braided layer 22, a first hot pressing is performed after forming the second braided layer 22, and a second hot pressing is performed after forming the third braided layer 22. In some embodiments, the first hot pressing is performed after forming the first braided layer 2, the second hot pressing is performed after forming the second braided layer 22, and the third hot pressing is performed after forming the third braided layer 22.

PAEK material is biologically inert and are difficult to fuse with bone tissue. In some medical devices that require bone fusion, it may be loosened due to poor bone fusion. To this end, the outer layer of the present disclosure may use a composite material containing bio-ceramics, or undergo surface treatment, to enhance cell affinity and bone fusion effect.

In some embodiments, the affinity layer 24 may further include bioceramics, which may include, for example, aluminum oxide, zirconium oxide, carbon biomaterials, calcium phosphate, bioglass, calcium sulfate, or a combination thereof. A layer of affinity layer 24 may contain about 0.5-5% by weight of bioceramics, such as about 1-4.5%, about 1.5%-4%, about 2-3.5%, about 2.5-3%, etc. The composite sandwich structure of orthopedic implant 100 of the present disclosure may include bioceramics or collagen coating only in the outermost affinity layer 24.

In some embodiments, the surface of the affinity layer 24 may be further subjected to surface treatment, such as radiation surface treatment, plasma surface treatment, collagen surface coating, chemical solution treatment, or a combination thereof. Among them, by plasma surface treatment, for example, highly activity free radicals or peroxide radicals may be generated on the surface, enabling better bonding with bioactive materials such as collagen.

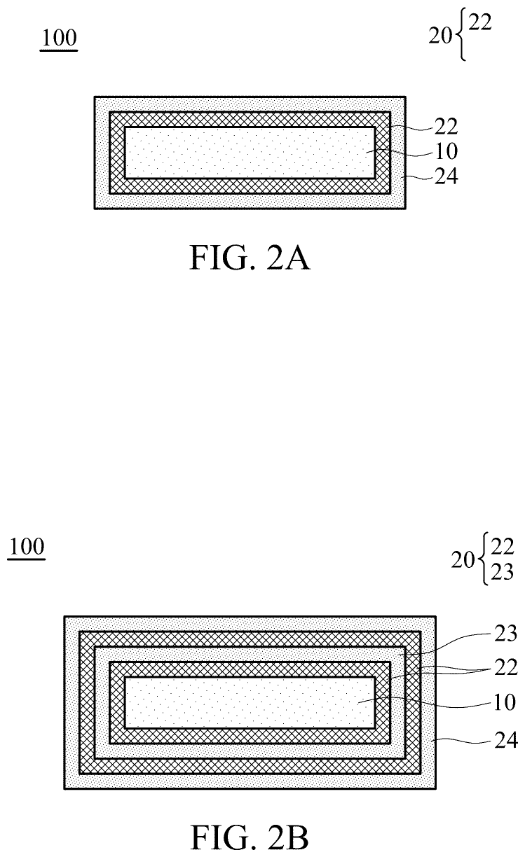

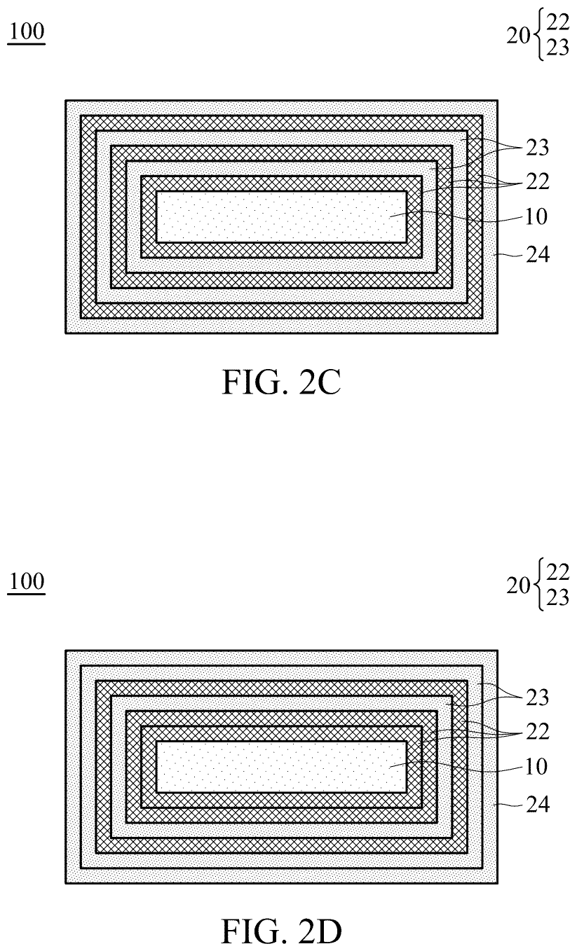

It should be noted that although FIG. 1A to FIG. 1D illustrate the cross-sectional shape of the core 10 as a circle, the cross-sectional shape of the core 10 of the present disclosure is not limited to a circle, and may also be an ellipse, a trapezoid, a triangle, a quadrilateral, a polygon, an irregular shape, etc. For example, referring to FIG. 2A-FIG. 2D, the core 10 may also have a rectangular cross-section, and the reinforcement layer 20 covering the core 10 may have one or more alternating or non-alternating braided layer 22 and structural layer 23. FIG. 2A only shows a single braided layer 22, which is conformably formed on the core with a rectangular cross-section. The materials and the forming method of the composite sandwich structure of orthopedic implant 100 shown in FIG. 2A-FIG. 2D may refer to the related description of the composite sandwich structure of orthopedic implant 100 shown in FIG. 1A to FIG. 1D, and will not be repeated here.

In summary, the present disclosure strengthens the PAEK material with a reinforcement layer having a carbon fiber bundle braided layer, and covers the carbon fiber bundle braided layer with an affinity layer of PAEK material as the outermost layer of the orthopedic implant, thereby forming an orthopedic implant that has both mechanical strength and ductility. In addition, since the carbon fiber bundle braided layer is a variable braided structure, the mechanical properties of different portions of the implant can be adjusted to meet requirements. In addition, the number and order of the carbon fiber bundle braided layer, the structural layer and the affinity layer can be adjusted to meet requirements. Therefore, the present disclosure may have a wide range of applications.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A composite sandwich structure of orthopedic implant, comprising:
a core, wherein a material of the core comprises polyaryletherketone (PAEK);
a reinforcement layer covering a surface of the core, wherein the reinforcement layer includes at least one braided layer composed of carbon fiber bundles; and
an affinity layer covering a surface of the reinforcement layer, wherein a material of the affinity layer includes polyaryletherketone.

2. The composite sandwich structure of orthopedic implant as claimed in claim 1, wherein at least one structural layer covering the at least one braided layer is further included in the reinforcement layer, and a material of the at least one structural layer includes polyaryletherketone.

3. The composite sandwich structure of orthopedic implant as claimed in claim 2, wherein the at least one braided layer covering a surface of the at least one structural layer is further included in the reinforcement layer.

4. The composite sandwich structure of orthopedic implant as claimed in claim 1, wherein the core further includes 0%-35% by weight of a carbon staple fiber.

5. The composite sandwich structure of orthopedic implant as claimed in claim 1, wherein the material of the affinity layer further includes 0.5%-5% by weight of a bioceramic.

6. The composite sandwich structure of orthopedic implant as claimed in claim 5, wherein the bioceramic includes alumina, zirconium oxide, carbon biomaterial, calcium phosphate, bioglass, calcium sulfate, and/or a combination thereof.

7. The composite sandwich structure of orthopedic implant as claimed in claim 1, wherein the affinity layer is treated by including radiation surface treatment, plasma surface treatment, collagen surface coating treatment, chemical solution treatment, and/or a combination thereof.
